(19) United States
(12) Patent Application Publication
(10) Pub. No.: US 2006/0106445 A1
(43) Pub. Date: May 18, 2006

(54) Method and apparatus for reliably placing and adjusting a left ventricular pacemaker lead

(76) Inventor: Ian Woollett, Norfolk, VA (US)

Correspondence Address: BARLOW, JOSEPHS & HOLMES, LTD., 101 DYER STREET, 5TH FLOOR, PROVIDENCE, RI, 02903, US

(21) Appl. No.: 11/272,634

(22) Filed: Nov. 14, 2005

Related U.S. Application Data
(60) Provisional application No. 60/628,444, filed on Nov. 16, 2004.

(51) Int. Cl. A61N 1/05

(52) U.S. Cl. 607/122

(57) ABSTRACT

A left ventricular stimulation lead construction and a technique for lead placement that provides for improved anchoring and positioning of the electrode located on the distal end of the lead, while also including a method for readjusting the positioning of the lead as necessary. The lead includes a small diameter guide wire wherein the distal end of the guide wire serves as an anchor for the pacing lead. The lead structure includes a central hollow lumen to allow passage of the guide wire and a locking mechanism positioned on the proximal end of the pacing lead to fix the ends of the guide wire and pacing lead relative to one another. The method provides for advancing the guide wire until it is anchored, positioning the pacing lead along the guide wire into the desired position relative to the cardiac tissue, releasably fixing the lead relative to the guide wire, wherein the guide wire is permanently retained to serve as an anchor.

### CROSS-REFERENCE TO RELATED APPLICATIONS

This application is related to and claims priority from earlier filed U.S. Provisional Patent Application No. 60/628,444, filed Nov. 16, 2004, the contents of which are incorporated herein by reference.

### BACKGROUND OF THE INVENTION

The present invention relates generally to a new apparatus and method for the placement of a left ventricular stimulation lead via the coronary sinus and associated branches of the cardiac venous system. More specifically, the present invention relates to a unique lead construction that includes an integrated guide wire, wherein the guide wire is permanently retained in position to allow accurate placement of the lead, improved anchoring of the lead and the ability to readjust lead positioning as necessary to avoid diaphragmatic pacing stimulation and to achieve the optimal pacing site for hemodynamic benefit.

It is common in the healthcare related field to utilize electrical stimulation of body tissue and organs as a method of treating various conditions. Such stimulation is generally delivered via an electrical lead that extends from a pulse generator device to a target stimulation site. The electrical leads are typically constructed to include one or more stimulation electrodes joined to a distal portion of the lead, wherein the distal portion of the lead and electrode(s) thereon are positioned and anchored in proximity to the target site. In this regard, various lead structures and methods for positioning and anchoring lead electrodes in proximity to target sites have been developed over the years. However, as the variety of possible therapy options expands, new structures and methods are necessary to anchor lead electrodes in order to accomplish the desired results associated with the emerging therapy delivery requirements.

For example, it is well known in the art that the use of a transvenous approach to implant a left ventricular pacing lead has dramatically increased the flexibility and number of treatment options available for many cardiac patients. In many cases, pacing the heart simultaneously from the left and right ventricles will allow restoration of a more synchronous pattern of contraction, which in turn can improve the efficiency of the cardiac cycle. Prior to the development of this technique, many patients with significant left ventricular dysfunction, who would benefit from the placement of a left ventricular pacing lead, generally required that a true epicardial pacing lead be surgically placed if any benefit was to be obtained. Even now, due to difficulties and limitations of the coronary sinus approach, it is often necessary to surgically implant a pacing lead after a failed transvenous attempt. Further, there have always been tradeoffs associated with the usage of transvenously placed leads in many patients. For example, like any foreign body introduced into the cardiovascular system, a transvenous cardiac lead presents an obstruction to the normal flow of blood. This obstruction can potentially interfere with the normal operation of one or more of the valves of the heart and may result in not only a diminished blood flow, but also may lead to the formation of microemboli. However, the benefits associated with the use of a transvenously placed left ventricular lead, as opposed to a surgically placed lead, generally include lower procedure related morbidity, lack of need for a second procedure, and likely better long-term lead survival.

A typical transvenous left ventricular lead such as provided in the prior art consists of an elongated tubular member that includes a proximal connector which is attached to a pulse generator device and a distally located electrode for transmitting signals generated by the pulse generator into the tissue to be stimulated. The distal electrode can either be single or dual and can offer either unipolar or bipolar pacing capabilities. In many cases, the lead is installed utilizing a technique known as “over-the-wire”, wherein the lead itself is formed to include a centrally disposed lumen through which a stylet or guide wire is inserted to manipulate and assist in directing the lead into the coronary sinus and down the desired venous branch. In this procedure, the guide wire is typically inserted transvenously, and the lead then extended along the guide wire until the desired placement of the lead is obtained. At the conclusion of the lead placement procedure, the guide wire is withdrawn and the proximate end of the lead is attached to the pulse generator.

The difficulty with this type of lead and the traditional “over-the-wire” placement method is that it is often difficult to properly anchor the lead into the desired location in one of the coronary venous branches. In particular, once the guide wire is removed, the potential exists for the lead to drift or draw back along the vessel into which it is placed, causing the lead to fall out and thereby requiring that the lead be reinserted.

Generally, in the prior art, only two options exist for resolving this lead drifting problem. The first option-is to extend the lead down the tapering vein until the distal end of the lead itself becomes wedged against the walls of the blood vessel (in some instances utilizing tines at the tip of the lead to engage the walls of the blood vessel and help hold it in place). This method often however often results in placing the pacing electrode that is located at the distal end of the lead in a position that may represent a poor compromise with regard to the more proximal sites that have been demonstrated to give superior hemodynamic benefits. In addition, the more distal sites often have a higher incidence of unintended stimulation of the diaphragm during pacing, a condition that can be very uncomfortable for the patent and may require subsequent lead repositioning or abandonment of left ventricular pacing altogether. The second option is to utilize a lead that itself includes a preformed curve or twist which is built into the structure of the distal end of the lead. During the placement procedure, the lead is maintained in a straight configuration by the use of a stiff guide wire. Upon removal of the guide wire, the distal end of the lead is allowed to return to its curved or twisted shape in a manner that hopefully serves to wedge the lead and maintain its position in a more proximal location. However, this method relies on having a properly sized vein to allow matching of the lead curve to the dimensions of the vein into which the lead is placed and often produces a less stable lead placement than a more distal “wedged” position. Currently, there are no active mechanical anchors in general use for coronary venous leads.

In addition, in order to maximize the degree of “resynchrony” and subsequent hemodynamic and clinical benefit, it is often desirable to place the pacing electrode in a more basal proximal location rather then a location further toward the apex of the heart. Frequently the venous branches at these more proximal locations are larger in diameter and are not optimal for lead stability. Therefore, it is often necessary to choose between a more apical lead location that is more secure but may offer less clinical benefit and a more proximal location that may not be secure but may offer more clinical benefit.

Accordingly, there is a need for a lead construction and technique for placing a transvenous left ventricular pacing lead via the coronary veins that provides for enhanced control over the lead positioning while also providing improved anchoring in various sized veins. Further, there is a need for a lead construction and placement method that facilitates optimal placement of the pacing electrode in a manner that maximizes hemodynamic benefit while allowing readjustment if needed to minimize diaphragmatic stimulation.

### BRIEF SUMMARY OF THE INVENTION

In this regard, the present invention provides for a novel left ventricular stimulation lead construction and a technique for lead placement that provides for improved anchoring and positioning of the electrode located on the distal end of the lead, while also including a method for readjusting the positioning of the lead as necessary.

In one aspect of the present invention, a transvenous left ventricular lead is provided that includes a small diameter guide wire wherein the distal end of the guide wire serves as an anchor for the pacing lead. In this regard, the pacing lead structure includes a central hollow lumen to allow passage of the guide wire and a locking mechanism positioned on the proximal end of the pacing lead. The locking structure serves to fix the ends of the guide wire and pacing lead relative to one another and ultimately serves to interconnect the pacing lead to the pulse generator that is also located at the proximal end of the lead.

In accordance with a second aspect of the present invention, a method is provided for the positioning of a left ventricular stimulation lead in a venous system and more particularly in cardiac veins. The method includes, directing a guide wire into the vascular system utilizing any of a number of commonly available guide catheters. After the guide wire has been advanced along the vascular system by a small distance, the pacing lead is then slid in a forward manner to follow the path established by the guide wire in the well known “over the wire” technique. This procedure is followed until the guide wire approaches the desired location for the placement of the pacing electrode. Once the desired position is reached, the guide wire is further extended, by advancing the guide wire into a small distal branch of the vascular system. The guide wire itself is then anchored in place by wedging the distal end of the guide wire in the small distal branch. The pacing lead is then slid along the guide wire into the exact position desired for placement of the pacing electrode relative to the cardiac tissue. In this manner, it can be seen that since the distal end of the guide wire is anchored, the positioning of the pacing electrode and the lead can be adjusted with great accuracy by sliding the pacing along the guide wire and using the guide wire as a fixed point of reference. This allows the lead to be brought to a more optimal proximal position without being dependant on the caliber of the vein in which the lead is being positioned.

By using the lead construction and lead placement method of the present invention, the pacing electrode can be placed optimally to achieve the required resynchronization while avoiding placement of the lead such that the diaphragm is stimulated. Further, the present invention allows optimal placement of the lead regardless of the particular diameter of the vein at the desired location. Additionally, with the distal end of the guide wire anchored in a remote and small vein, the lead placement will achieve improved long-term stability while also allowing repositioning of the lead along the length of the guide wire via the undoing the locking mechanism if relocation of the lead becomes necessary at a later time.

Accordingly, it is an object of the present invention to provide a left ventricular cardiac pacing lead that includes integrated anchoring means that extends beyond the desired position for the pacing electrode in a manner that allows anchoring of the lead in smaller distal branches of the vascular system. It is a further object of the present invention to provide a left ventricular pacing lead that includes anchoring means that extends beyond the desired position for the pacing electrode in a manner that allows both anchoring of the lead in smaller branches of the vascular system while also allowing reliable and accurate adjustment of the pacing lead relative to the anchoring means. It is still a further object of the present invention to provide a method for the placement of a left ventricular pacing lead that includes anchoring a guide wire in smaller branches of the vascular system while allowing placement of a pacing lead at a more proximal and more advantageous location that both maximizes hemodynamic benefit while allowing readjustment if needed to minimize diaphragmatic stimulation

These together with other objects of the invention, along with various features of novelty, which characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

The following drawings are illustrative of the best mode presently contemplated for carrying out the present invention and therefore do not limit the scope of the invention, but are presented to assist in providing a proper understanding of the invention. The drawings are not to scale (unless so stated) and are intended for use in conjunction with the following detailed description. Accordingly, the present invention will hereinafter be described in conjunction with the appended drawings, wherein like numerals denote like elements, and:

FIG. 1 is a schematic illustration of an implanted medical device in the form of a pacemaker and the associated electrical medical lead;

FIG. 2 illustrates the electrical medical lead as it is implanted in the coronary sinus;

FIG. 3A schematically illustrates a cross-sectional view of the distal end of the guide wire and the over-the wire lead;

FIG. 3B schematically illustrates a cross-sectional view of the locking means, the proximal end of the guide wire and the over-the wire lead;

FIG. 4A is a schematic diagram illustrating the implantation of the guide wire of the lead of FIG. 2; and

FIG. 4B is a schematic diagram illustrating the positioning of the over-the wire lead relative to the guide wire.

### DETAILED DESCRIPTION OF THE INVENTION

Now referring to the drawings, as was stated above, the present invention provides for a novel left ventricular stimulation lead construction and a technique for lead placement that provides for improved anchoring and positioning of the electrode located on the distal end of the lead, while also including a method for readjusting the positioning of the lead as necessary. It can be appreciated by one skilled in the art of implantable medical devices that, while embodiments of the present invention are described herein in the context of an epicardial implant via a cardiac vein, embodiments of the present invention may be implemented in a host of other therapy delivery contexts.

The present invention is directed to a lead that is implanted through a cardiac venous system, such as the coronary sinus and its tributaries and is utilized to stimulate, defibrillate, and/or sense the atrium and ventricle of the left side of the heart. As was stated above, in the prior art it has been difficult to reliably implant leads within the coronary sinus. For example, a typical coronary sinus is 10 millimeters at its largest diameter (near the outflow to the right atrium) and narrows until it has a diameter of between approximately 2-3 millimeters and merges to the great cardiac vein. Thus any leads having larger sizes could be expected to diminish the flow of blood through the coronary sinus. For leads placed directly within the cardiac chambers, there have been developed a wide variety of fixation mechanisms including tines and actively extending screws that dig into the myocardium. These are all predicated on the ability to position the tip of the lead directly up against a surface to attach to which is not available in a cardiac vein. In addition, concerns about bleeding caused by perforation of the thin walled cardiac veins have limited interest in such active mechanisms. The fixation of a lead within the cardiac vein is further complicated by the fact that, unlike a heart chamber where the fibrotic tissue response is used to assist lead fixation, no such fibrotic response can be reliably expected in the vein. Thus, no fibrotic tissue response is available to assist in lead fixation. Moreover, there are a wide range of variations among patients in the anatomy of the coronary sinus, cardiac veins, and the heart, therefore creating a need to accommodate these variations by allowing for adjustments in the spacing between the anchoring point of the lead and the placement of the pacing electrode. Thus, the present invention is directed to a single lead with an electrode that is selectively positionable relative to the anchoring point of the lead that is particularly suitable for placement along the left side of the heart through the coronary sinus and its tributaries with a high degree of efficiency and accuracy.

Further, while illustrative embodiments of the invention are described below, in the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

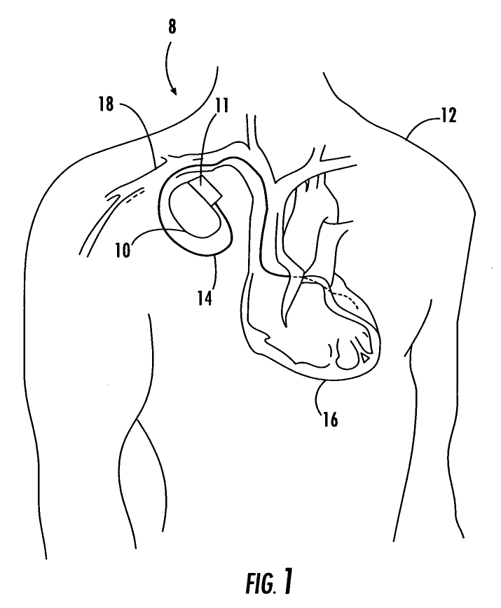

FIG. 1 illustrates an implantable medical device (IMD) system 8, which includes an implantable electronic device 10, such as a pacemaker, defibrillator, or the like, that has been implanted in a patient 12. The device 10 is housed within a hermetically sealed, biologically inert outer canister or housing, which may itself be conductive so as to serve as an electrode in the pacemaker's pacing/sensing circuit. At least one electrical lead 14 is electrically coupled to the pacemaker 10 in a conventional manner and extends into the patient's heart 16 via a vein 18. The details of the lead 14 structure and method of implantation will be discussed in further detail below.

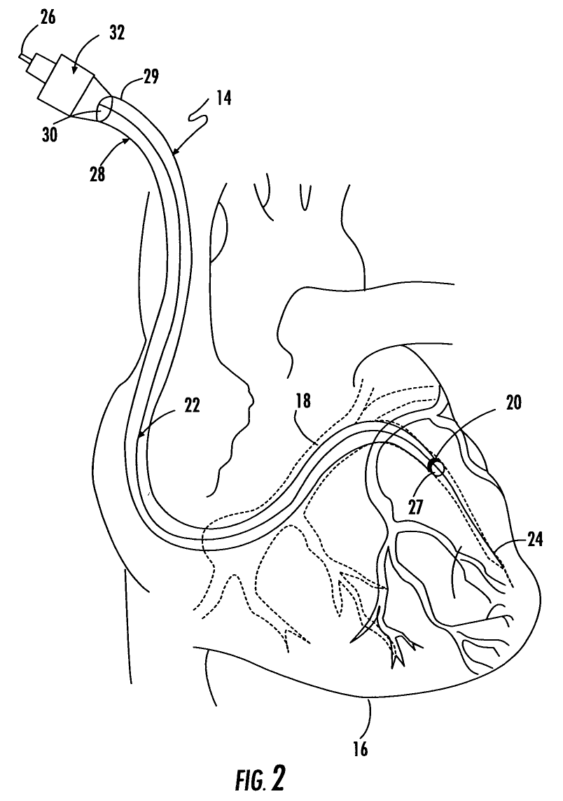

Referring to FIG. 2, generally the lead 14 includes at least one exposed conductive electrode 20 for sensing cardiac activity, delivering electrical pacing stimuli to the heart 16, or providing a stimulating voltage to defibrillate the heart 16. The lead 14 may be implanted with its electrode 20 positioned adjacent the atrium or the ventricle, or both, of the heart 16.

In the embodiment illustrated herein, some variations in the structure, construction, and/or location of the lead 14 may be useful in adapting the lead 14 to its intended use. That is, while the lead 14 of the present invention is particularly suited for left ventricular pacing, the construction and/or positioning of the lead 14 may be altered when the lead 14 is used to variously provide left ventricle and atrial pacing, coronary sinus defibrillation, and/or left ventricle defibrillation. Further, while the process used to position the lead 14 for use in each of these applications may differ in some manner, in general practice for each application implantation is relatively similar.

Turning again to FIG. 2, the lead 14 of the present invention is depicted in its implanted position as a transvenous left ventricular pacing lead. The lead 14 of the present invention generally includes a small diameter guide wire 22 having a distal end 24 and a proximate end 26, wherein the distal end 24 of the guide wire 22 serves as an anchor for the pacing lead 14. The electrical pacing lead 14 structure is formed as an elongate flexible body 28 having a distal end 27 and a proximate end 29 and includes a central hollow lumen 30 extending from the distal end 27 to the proximate end 29 to allow passage of the guide wire 22 therethrough. The electrical pacing lead 14 includes at least one electrode 20 adjacent the distal end 27 thereof and further includes a locking mechanism 32 that is positioned on the proximal end 28 thereof. The locking mechanism 32 serves to fix the proximal ends 26, 29 of the guide wire 22 and pacing lead 14 relative to one another and ultimately serves to interconnect the pacing lead 14 to the pacemaker 10 that is also located at the proximal end 29 of the lead 14.

The guide wire 22 of the present invention is an elongate flexible member that preferably takes the form of a fine filament. The guide wire 22 may be constructed from a variety of materials including a metallic material such as stainless steel wire, a polymer filament material or a glass fiber. While the guide wire 22 that is utilized in the present invention may in fact be a currently existing guide wire, preferably the ideal guide wire 22 would be designed specifically for this use and would likely consist of a soft body, a stiffer distal section, and a soft non-traumatic tip or a tip with a preformed curve, corkscrew or other anchoring mechanism. Further, the guide wire 22 may be constructed in a manner that provides for varying flexibility along its length either by varying its material, thickness, or modulus of elasticity. For example, the guide wire 22 may taper or have a diameter that is graduated between its proximal end 26 and its distal end 24 wherein the resultant distal end 24 has a diameter that is either larger or smaller than that of the proximal end 26. Similarly, the guide wire 22 may be constructed from varying materials that have more flexible modulus of elasticity at the proximal end 26 graduating to a stiffer modulus of elasticity toward the distal end 24 before softening again for an atraumatic tip. In either case, the purpose is to form a guide wire 22 that can be directed through the veins 18 of the coronary sinus as is well known in the art and be anchored into the veins 18 in accordance with the method of the present invention. This would minimize the risk of damage to the small veins while providing optimal anchoring capabilities.

Alternately, it may prove optimal to initially utilize a commercially available guide wire 22 to direct the lead 14 to the desired venous branch and to then swap it out for a permanent guide wire 22 with more desirable anchoring characteristics.

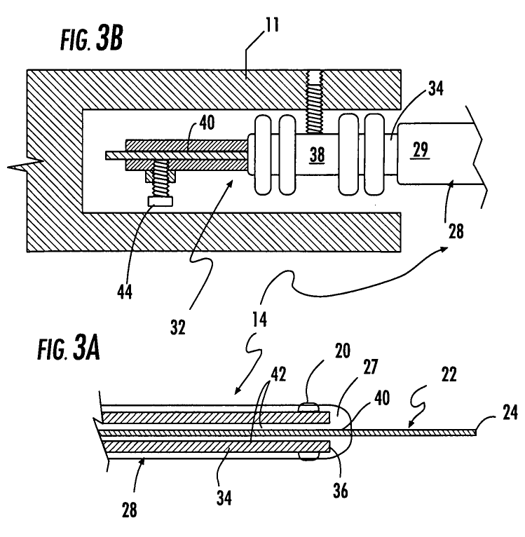

Turning now to FIGS. 3A and 3B the electrical pacing lead 14 of the present invention includes an elongated tubular body 28 that is sufficiently flexible to allow the lead 14 to traverse the tortuous path of the cardiac venous system. Preferably, the tubular body 28 is formed from a silicone rubber, polyurethane, or other implantable material, such as a polymer. The lead 14 further includes an electrically conductive pathway 34 therein. The electrically conductive pathway 34 is comprised of a flexible conductor, such as a metallic wire encased in an insulative material, such as polyurethane, silicone, or flouropolymer. An electrode 20 is positioned adjacent the distal end 27 of the lead 14 and is exposed to the outer surface of the tubular body 28. The electrode 20 is coupled to the distal end 36 to the flexible conductor 34 such that they are electrically coupled together. Thus, an electrical signal, such as a pacing signal, applied to the proximal end 38 of the flexible conductor 34 ultimately passes to the electrode 20, causing the adjacent chamber of the heart to contract. The lead 14 further includes a lumen 40 extending therethrough from the proximal end 29 to the distal end 27 through which the guide wire 22 passes. The lumen 40 is preferably formed to have a wall structure 42 that serves to electrically isolate the electrically passive guide wire 22 from the electrically conductive pathway 34. As can be seen, the guide wire 22 extends out of the distal end 27 of the lead 14 so that the guide wire 22 can be extended further into the coronary sinus for anchoring as described above.

FIG. 3B shows a cross sectional view of the guide wire 22 and the lead 14 at their proximal end portions and includes a schematic depiction of the locking means 32. The locking means 32 serves both as a means for connection to a socket 11 provided on the implantable device 10 as well as a locking mechanism that serves to fix the proximal ends of the guide wire 22 and the pacing lead 14 relative to one another. In particular, a possible mechanism for electrically coupling the proximate end 38 of the electrically conductive pathway 34 to the implantable device 10 and for locking the lead 14 relative to the guide wire 22 is shown. Preferably, a conventional IS-1 connector, such as is well known in the art, is modified in accordance with the present invention for use as the locking mechanism 32. The IS-1 connector 32 is coupled to the proximate end 29 of the tubular body 28 and extends around the lead 22. By utilizing the known IS-1 connector as both the locking mechanism and the lead attachment plug, the lead 14 of the present invention can be utilized without requiring large changes in the existing pulse generators in the industry. Currently the IS-1 connector 32 is connected onto the pacing lead 14 and is secured into contact with the plug 11 on the implanted device 10 to provide electrical interconnectivity between the pacing electrode 20 and the pulse generator 10. The present invention further provides for the addition of a locking screw 44 or clamping device to allow the lead 14 to be anchored to the guide wire 22 thereby preventing their movement relative to one another.

It is further anticipated that the implementation of the present invention may be accomplished either through an entirely new lead construction or via a modular attachment that would replace the standard IS-1 plug and serve to anchor the guide wire to a currently existing pacing lead while also providing an electrically conductive pathway to connect the pacing lead to the pulse generator. Finally, it is within the scope of the invention that the mechanism to fix the retained guide wire to the lead could be integrated into the lead itself rather then being part of the pulse generator header.

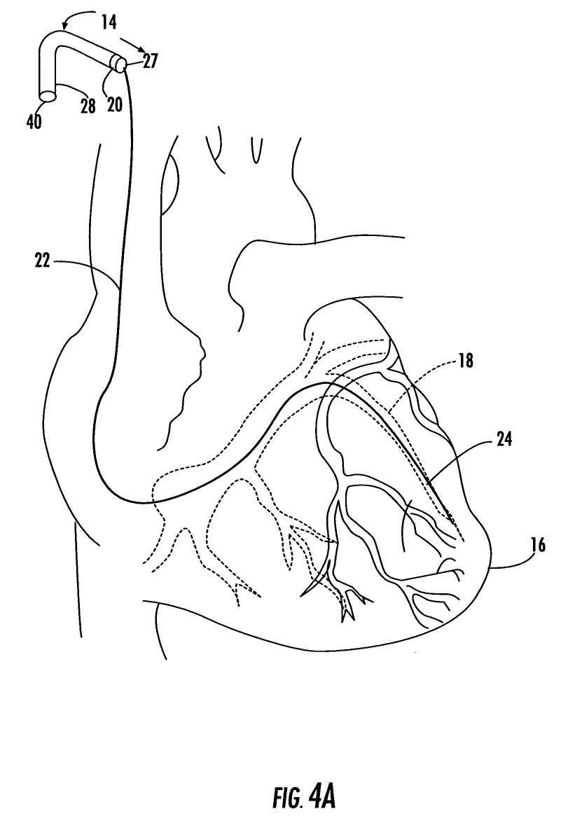

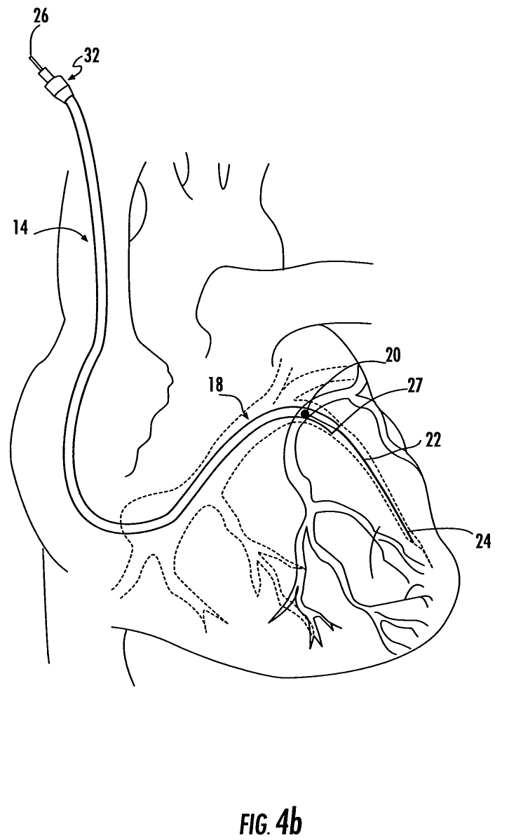

Turning now to FIGS. 4A and 4B, the method of the present invention is schematically illustrated. To the extent that certain components and procedures referenced herein are conventional in their design and operation, such components/procedures will not be described herein in detail, as it is believed that design and implementation of such components and the performance of such methods would be a matter of routine practice to those of ordinary skill in the art. For example, various processes for passing a catheter lead through the tortuous path of a representative cardiac venous system is well known in the art.

Generally, as illustrated in FIG. 4A, the guide wire 22 is inserted into the coronary sinus and extended along the venous pathway 18 into a position along the left side of the heart 16. The distal end 24 of the guide wire 22 is extended into the smaller venous branches until a suitable location is found for anchoring the distal end 24 of the guide wire 22. Next, an over-the wire lead 14 is extended along the guide wire 22. In a similar fashion, the above steps may also be accomplished by incrementally advancing the guide wire 22 and then the lead 14 in a repetitive process until the lead 14 reaches its desired location wherein the guide wire 22 is then further advanced into a suitable anchoring position. With the guide wire 22 anchored and the lead 14 at least partially inserted, the position of the lead 14 is adjusted relative to the guide wire 22 in order to position the pacing electrode 20 on the distal end 27 of the lead 14 at the desired location within the coronary sinus. Finally, the proximate end 29 of the lead is releasably locked relative to the proximate end 26 of the guide wire 22 to prevent movement of the lead 14 relative to the guide wire 22. This is accomplished by tightening the locking means, such as the setscrew 44 or clamping device, located in the connector 32 at the proximate end 29 of the lead 14. Unlike all current techniques, the guide wire 22 is therefore permanently retained and is able to serve as a distal anchor as well as a rail along which future repositioning of the lead 14 may be accomplished.

It should be appreciated by one skilled in the art that, while certain sizes, material selections and specifications have been described herein, the present invention lies in the primary concept of inserting and anchoring a guide wire around which a pacing lead is positioned wherein the pacing lead is then locked in position relative to the guide wire which is itself permanently retained. The materials described herein are for the purpose of illustration of the general concept only. Further, while there is shown and described herein certain specific structures embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A medical electrical lead, comprising:
a guide wire having a proximate end and a distal end, said distal end anchorable in the coronary sinus;
a lead having a proximate end and a distal end, said lead coupled with and movable along said guide wire, said lead having at least one electrode located proximate said distal end, wherein the position of said electrode may be varied relative to said distal end of said guide wire; and
locking means coupled to said proximate end of said lead, said locking means selectively engagable with said guide wire to prevent movement of said lead relative to said guide wire.

2. The medical electrical lead of claim 1, wherein said guide wire is constructed using a material selected from the group consisting of: metal wire, polymer strand and glass fiber.

3. The medical electrical lead of claim 1, wherein said distal end of said guide wire is configured to extend into and be anchored within a small diameter vein of the coronary sinus.

4. The medical electrical lead of claim 1, wherein the diameter of said guide wire varies between said proximal end and said distal end.

5. The medical electrical lead of claim 1, wherein the stiffness of said guide wire varies between said proximal end and said distal end.

6. The medical electrical lead of claim 1, wherein said lead is an over-the-wire lead.

7. The medical electrical lead of claim 1, wherein said lead includes an elongated flexible body extending between said proximate and distal ends, said elongated flexible body having a passage extending therethrough, wherein said guide wire passes through said passage.

8. The medical electrical lead of claim 1, wherein said lead includes at least one electrically conductive pathway extending from said proximal end to said electrode located on said distal end.

9. The medical electrical lead of claim 8, wherein said lead includes a core of electrically conductive material surrounded by a layer of insulative material.

10. The medical electrical lead of claim 8, wherein said locking means includes an electrical connector therein, said electrical connector in electrical communication with said electrically conductive pathway, said connector configured to interface with a signal generating means.

11. The medical electrical lead of claim 10, wherein said electrode is implemented for left ventricular pacing.

12. A method for positioning a medical electrical lead in a coronary sinus, comprising:
inserting a guide wire into the coronary sinus;
anchoring a distal end of said guide wire on the interior of a vein within the coronary sinus, wherein said guide wire is permanently retained within said vein;
guiding an over-the-wire lead along said guide wire and into the coronary sinus, said lead having at least one electrode located proximate a distal end thereof;
adjusting the position of said lead relative to said guide wire to place said electrode at the desired location within said coronary sinus; and
releasably locking a proximate end of said lead relative to a proximate end of said guide wire to prevent movement of said lead relative to said guide wire.

13. The method of claim 12, wherein the diameter of said guide wire varies between said proximal end and said distal end.

14. The method of claim 12, wherein the stiffness of said guide wire varies between said proximal end and said distal end.

15. The method of claim 12, wherein said lead comprises
an elongate flexible body having a proximate end and a distal end, said body having a core of electrically conductive material surrounded by a layer of insulative material;
a passage extending through said body between said proximate and said distal ends, wherein said guide wire passes through said passage;
at least one electrode located proximate said distal end; and
locking means coupled to said proximate end of said lead, said locking means selectively engagable with said guide wire to prevent movement of said lead relative to said guide wire.

16. The method of claim 12 wherein said electrode is implemented for left ventricular pacing.
